(19) United States
(12) Patent Application Publication
(10) Pub. No.: US 2002/0123615 A1
(43) Pub. Date: Sep. 5, 2002

(54) Quality improvement for animal whole blood protein products

(76) Inventor: John H. Lee, Olathe, KS (US)

Correspondence Address: John H. Lee, 1441 N. Lucy Montgomery Way Street, Olathe, KS, 66061, US

(21) Appl. No.: 09/753,088

(22) Filed: Jan. 2, 2001

(51) Int. Cl. A01N 1/02; C07K 1/00; C07K 14/00; C07K 16/00; C07K 17/00; A61K 35/14; A61K 38/16

(52) U.S. Cl. 530/380; 424/529; 435/2

(57) ABSTRACT

The micro counts, smell, color and contamination are major quality concerns for animal whole blood protein products. A method for improving the quality of animal whole blood protein products with heat and 0.2 to 3% hydrogen peroxide is provided. The inexpensive and simple method may be commercialized for bulk production. The processed products have mild smell, low micro counts, light color, decontamination, maximum protein recovery and similar nutrients comparing with the initial whole blood protein products. Also hydrolysis process may be used to hydrolyze the protein products to have higher water solubility.

### BACKGROUND OF THE INVENTION

Animal blood products are very good sources for proteins, which contain about 70-75% red blood cells and 25-30% plasma. Red blood cell contains about 92% protein and plasma contains about 78% protein. Plasma products have light color and are separated from red blood cells by a centrifugation process. Animal blood products with red blood cells have limited applications in the formulation of food, animal milk replacer, feed, pet food and other products. One major issue is the intense color. Also high micro counts and strong blood flavors are other two concerns.

Over the years, various attempts have been made to remove the intense color from animal blood products.

U.S. Pat. No. 5,880,266 discloses a process for removing the heme group from the globin fraction by water dilution, pH change, ultrafiltration and pH reverse.

U.S. Pat. No. 5,151,500 discloses a decolorized process by treating the blood to low pH. The iron is removed by a centrifugation process. Then the supernatant is treated with an oxidizing agent for the decolorization.

U.S. Pat. No. 5,108,908 discloses a decolorized process by treating blood to low pH. The heme with iron is removed by a centrifugation process.

U.S. Pat. No. 4,330,463 discloses a process to remove the heme from animal red blood cells by solvent extraction with the organic solvents such as methanol/ethanol/water mixture, recovering the blood cell protein isolate from the extraction residue.

U.S. Pat. No. 4,180,592 discloses a decolorization process by a reversal pH or heat and an excess oxidizing agent, after which the excess oxidizing agent is removed by adding a further amount of blood. The color is still red by the further amount of blood.

U.S. Pat. No. 4,098,780 discloses a method of separating iron compounds from protein, mainly globin, with ethanol, pH change and centrifuge processes.

European Patent 0 460 219 discloses a process by treating washed animal red blood cells under alkaline conditions and treating the resulting product under oxidizing conditions.

These patents and other references can be used for the decolorization of animal blood products. However the processes may involve several process steps or need high cost equipment, which increase the process cost. Protein recovery may be affected by these separation and extraction processes. Also the heme by-product is another issue. There are still no the decolorized animal blood products in the commercial market. There is a need for an inexpensive and simple process by which animal blood can be decolorized to have good products with maximum protein recovery and lighter color. Then the commercialized products may be available.

The purpose of the present invention is to provide new decolorized process for animal blood protein products with maximum protein recovery and without blood intense color through a simple process at low cost. Also the micro counts and product flavor can be improved by the process.

### SUMMARY OF THE INVENTION

The present invention overcomes the above problems and provides decolorized animal blood products which retain the iron and protein of the starting blood compositions. The invention also provides the method for forming these products.

The products are formed by decolorizing a starting composition comprising a blood component, which includes heme from whole animal blood or red blood cell fraction. The blood components can be obtained from any source, including swine, cattle, turkey, chicken, goat and other animals.

This decolorized process is carried out by heating animal blood composition to a temperature above 60° C. for a period of time such as from 2 to 30 minutes and then by mixing less than 3% hydrogen peroxide against animal blood composition by weight or volume base. For example, 1% of 100% hydrogen peroxide can be converted to the equivalent about 3% of 33% hydrogen peroxide solution to take into account commercially available hydrogen peroxide solution of varying concentrations of hydrogen peroxide. Conventional drying means can be used to yield a powder or particle dry form having a light color and a mild flavor. If a highly soluble product is desired, the decolorized liquid product can be hydrolyzed by a hydrolysis process. The value of pH can be adjusted to certain level for optimized process.

As a result of the processing conditions, the processes do not result in the formation of any by-product such as the heme. Therefore, the decolorized products of the invention will retain all protein and iron with 100% recovery if there is no processing loss. In most cases, the decolorized products will retain at least 85% of the protein and iron.

The quantity of hydrogen peroxide remaining in the dry decolorized products, without any treatment to remove excess hydrogen peroxide, is very minimized because hydrogen peroxide is used at about 1% level in most cases, which is reacted with animal blood. It was undetectable in our examples.

The products have very low bacterial counts such as E. Coli, Salmonella and Coliforms because of the treatment with hydrogen peroxide. Finally, the product flavors are mild without strong animal blood flavors.

### DETAILED DESCRIPTION OF THE PREFERRED EMBODIMENTS

The following examples set forth preferred methods in accordance with the invention. It is to be understood, however, that these examples are provided by way of illustration and nothing therein should be taken as a limitation upon the overall scope of the invention.

### EXAMPLE 1

Chicken whole blood was heated to 78° C. and the temperature was maintained for 6 minutes. Hydrogen peroxide (1.0% (w/v) against the blood volume) was added while agitating the solution. The color change was monitored while the solution was mixed for 20 minutes. During this time, the color changed from blood red to a yellowish color.

### EXAMPLE 2

Bovine red blood cell fraction separated from the plasma was diluted with water at 1:3 (volume base). The solution was heated to 68° C. and the temperature was maintained for 5 minutes. Hydrogen peroxide (0.5% (v/v) against the total volume) was added while agitating the solution. The color change was monitored while the solution was mixed for 15 minutes. During this time, the color changed from blood red to a yellowish color.

### EXAMPLE 3

Porcine red blood cell fraction was heated to 78° C. under an agitation and was mixed for 5 minutes. Hydrogen peroxide (1.6% (w/w) against the blood weight) was added while agitating the solution. The color change was monitored while the solution was mixed for 10 minutes forming a protein slurry having small particles. During this time, the color changed from blood red to a yellowish color. Then the product was dried. The product was tested with 0.01 M KMnO4 against 5% product solution. There was no detectable hydrogen peroxide remaining in the product. The analytical data for the powder product were: crude protein (91.5%), acid hydrolysis fat (1.72%) and moisture (7.76%).

### EXAMPLE 4

Turkey whole blood was diluted with water at 1:1 (volume base). The solution was heated to 70° C. and the temperature was maintained for 6 minutes. Hydrogen peroxide (0.6% (v/v) against the total volume) was added while agitating the solution. The color change was monitored while the solution was mixed for 20 minutes. During this time, the color changed from blood red to a yellowish color. A proteolytic enzyme was added at a rate of 3 g of enzyme per kg of protein. The resulting mixture was maintained at a temperature 60° C. for 6 hours. The pH was changed to 8.5-9.5 at every hour. The final solution was dried at 98° C. The solid was ground into yellowish powder. The dry product was tested with 0.01 M KMnO4 against 5% product solution. There was no detectable hydrogen peroxide remaining in the product. The analytical data for the powder product were: crude protein (87.6%), acid hydrolysis fat (0.85%) and moisture (7.53%).

### EXAMPLE 5

Porcine whole blood was heated to 75° C. under an agitation and was mixed for 6 minutes. Hydrogen peroxide (1.1% (w/w) against the blood weight) was added while agitating the blood. During the mixing time of 20 minutes, the color changed from blood red to a yellowish color. The product was dried. The dry product was tested with 0.01 M KMnO4 against 5% product solution. There was no detectable hydrogen peroxide remaining in the product. The analytical data for the product were: crude protein (88.0%), acid hydrolysis fat (0.75%), moisture (4.53%), acid detergent fiber (0.37%), ash (6.88%), sodium (1.65%), iron (2135 ppm), standard plate count (5000 cfu/g), E. Coli (<3 cfu/g), Salmonella (negative/25 g) and Coliforms (<3 cfu/g).

Inventors: Lee; John H., 1441 N. Lucy Montgomery Way Street, Olathe, Kans. 66061 (US)

Assignee: Rigel Technology Corporation, 14440 W. 100 Street, Lenexa, Kans. 66215

U.S. Class: 530/385; 426/32; 435/68; 260/112

Field of Search: 530/385; 426/32; 435/68; 260/112

### REFERENCE CITED

U.S. Patent Documents

| | | | |
|---|---|---|---|
| 4098780 | July, 1978 | Lindroos et al. | 260/112. |
| 4180592 | Dec., 1979 | Buckley et al. | 426/32. |
| 4330463 | May, 1982 | Luijerink | 260/112. |
| 5108908 | April, 1992 | Coves et al. | 435/68. |
| 5151500 | Sep., 1992 | Wismer-Pedersen et al. | 530/385. |
| 5880266 | March, 1999 | De Buyser et al. | 530/385. |

Foreign Patent Documents

| | | |
|---|---|---|
| 0 460 219 | Dec., 1991 | EP |

What is claimed is:

1. A method of preparing improved animal whole blood protein products comprising the steps of:
(a). heating an animal whole blood product to a temperature above 58° C. for a period of time to inactive the peroxidase in the blood products;
(b). mixing 0.2% to 3% by weight or volume of an oxidant with the composition resulting from step (a) to yield the improved products.

2. The method of claim 1, wherein said oxidant is hydrogen peroxide.

3. The method of claim 1, further including the step of hydrolyzing the resulting product from said mixing step (b) so as to increase the water solubility of said product. Also high heat may be applied for the hydrolyzed product.

4. The method of claim 1, wherein the pH may be changed for optimized process.

5. A improved animal blood protein product with mild smell, light color, low micro counts, decontamination, maximum protein recovery and similar nutrients is prepared by the method of claim 1.
